(19) United States
(12) Patent Application Publication
(10) Pub. No.: US 2022/0395326 A1
(43) Pub. Date: Dec. 15, 2022

(54) INTRODUCER FOR ELECTROSURGICAL INSTRUMENT

(71) Applicant: Creo Medical Limited, Chepstow (GB)

(72) Inventors: Christopher Paul Hancock, Chepstow (GB); Patrick Bum, Chepstow (GB); Craig Jonathan Gulliford, Chepstow (GB)

(73) Assignee: Creo Medical Limited, Chepstow (GB)

(21) Appl. No.: 17/773,012

(22) PCT Filed: Sep. 2, 2020
(86) PCT No.: PCT/EP2020/074385

§ 371 Date: Apr. 28, 2022

(30) Foreign Application Priority Data
Nov. 1, 2019 (GB) 1915898.9

(51) Int. Cl. A61B 18/18

(52) U.S. Cl. CPC A61B 18/1815; A61B 2018/00017

(57) ABSTRACT

Various embodiments provide an introducer for introducing an electrosurgical instrument into a body of a patient. The introducer comprises: a tubular member defining a lumen through which the electrosurgical instrument is insertable; and a cooling assembly configured to remove heat from the tubular member. Other embodiments provide an electrosurgical system comprising an electrosurgical instrument and an introducer.

### FIELD OF THE INVENTION

The invention relates to an introducer for introducing an electrosurgical instrument into a body of a patient. The introducer may be used to perform percutaneous electrosurgical procedures, as well electrosurgical procedures involving a surgical scoping device.

### BACKGROUND

Electromagnetic (EM) energy, and in particular microwave and radiofrequency (RF) energy, has been found to be useful in electrosurgical operations, for its ability to cut, coagulate, and ablate body tissue. Typically, apparatus for delivering EM energy to body tissue includes a generator comprising a source of EM energy, and an electrosurgical instrument connected to the generator, for delivering the energy to tissue. Conventional electrosurgical instruments are often designed to be inserted percutaneously into the patient's body. However, it can be difficult to locate the instrument percutaneously in the body, for example if the target site is in a moving lung or a thin walled section of the gastrointestinal (GI) tract. Other electrosurgical instruments can be delivered to a target site by a surgical scoping device (e.g. an endoscope) which can be run through channels in the body such as airways or the lumen of the oesophagus or colon. This allows for minimally invasive treatments, which can reduce the mortality rate of patients and reduce intraoperative and postoperative complication rates.

Tissue ablation using microwave EM energy is based on the fact that biological tissue is largely composed of water. Human soft organ tissue is typically between 70% and 80% water content. Water molecules have a permanent electric dipole moment, meaning that a charge imbalance exists across the molecule. This charge imbalance causes the molecules to move in response to the forces generated by application of a time varying electric field as the molecules rotate to align their electric dipole moment with the polarity of the applied field. At microwave frequencies, rapid molecular oscillations result in frictional heating and consequential dissipation of the field energy in the form of heat. This is known as dielectric heating. Water (a major component of blood) has a much higher dipole moment than fatty tissue and so for the same electric field, the heating of the water molecules in blood will occur more rapidly than the heating of the fat molecule.

This principle is harnessed in microwave ablation therapies, where water molecules in target tissue are rapidly heated by application of a localised electromagnetic field at microwave frequencies, resulting in tissue coagulation and cell death. It is known to use microwave emitting probes to treat various conditions in the lungs and other organs. For example, in the lungs, microwave radiation can be used to treat asthma and ablate tumours or lesions.

RF EM energy can be used for cutting and/or coagulation of biological tissue. The method of cutting using RF energy operates based on the principle that as an electric current passes through a tissue matrix (aided by the ionic contents of the cells), the impedance to the flow of electrons across the tissue generates heat. When a pure sine wave is applied to the tissue matrix, enough heat is generated within the cells to vaporise the water content of the tissue. There is thus a large rise in the internal pressure of the cell that cannot be controlled by the cell membrane, resulting in the cell rupturing. When this occurs over a wide area it can be seen that tissue has been transected.

One challenge facing the delivery of microwave and/or RF energy to treatment sites located within the body is how to prevent unwanted effects caused by losses from a cable that conveys the microwave energy to the treatment site. These losses often manifest as heating of the cable, which in turn can heat and potentially damage surrounding biological tissue.

### SUMMARY OF THE INVENTION

At its most general, the present invention provides an introducer for introducing an electrosurgical instrument into a body of a patient, where the introducer includes a cooling assembly configured to remove heat from the introducer. During operation of the electrosurgical instrument, the electrosurgical instrument may heat up, e.g. due to losses in a transmission line (or cable) of the instrument which is used to convey microwave and/or RF energy to a radiating tip of the instrument. When the electrosurgical instrument is introduced into the body of the patient via the introducer of the invention, the cooling assembly may be used to effectively remove heat generated by the electrosurgical instrument. The introducer may also act as a low temperature barrier between the electrosurgical instrument and the patient, such that the introducer may serve to reduce transmission of heat from the electrosurgical instrument to the patient. In this manner, it may be possible to avoid heating tissue along a length of the electrosurgical instrument, to avoid tissue damage outside of a target treatment area.

According to a first aspect of the invention, there is provided an introducer for introducing an electrosurgical instrument into a body of a patient, the introducer comprising: a tubular member defining a lumen through which the electrosurgical instrument is insertable; and a cooling assembly configured to remove heat from the tubular member.

The tubular member may be any suitable hollow member defining a lumen for receiving the electrosurgical instrument. The tubular member may, for example, have a substantially cylindrical shape. The lumen may be a channel defined within the tubular member along a length of the tubular member, which is dimensioned to receive the electrosurgical instrument.

The lumen may be dimensioned such that, when the electrosurgical instrument is inserted through the lumen, an outer surface of the electrosurgical instrument is in contact with an inner surface of the tubular member. For example, a cross-sectional area of the lumen may be arranged to substantially match a cross-sectional area of the electrosurgical instrument. This may ensure that, when the electrosurgical instrument is inserted into the lumen, heat generated by the electrosurgical instrument may be transferred to the tubular member. In some cases, the lumen may be dimensioned to form an interference fit with the electrosurgical instrument when the electrosurgical instrument is inserted into the lumen.

In use, the electrosurgical instrument may be introduced into the body of the patient via the introducer. The electrosurgical instrument may be inserted through the lumen in the tubular member, until a radiating tip of the electrosurgical instrument protrudes beyond a distal end of the tubular member and reaches a target treatment site.

The tubular member may be configured to be percutaneously inserted into the body of a patient. In such a case, the introducer may first be percutaneously inserted into the body of the patient. Then, the electrosurgical instrument may be inserted through the lumen of the tubular member, until the radiating tip of the electrosurgical instrument reaches a target treatment site within the patient.

In some cases, the tubular member may be configured to be inserted into the body of a patient via a scoping device, such as a laparoscope. In such a case, the tubular member may be dimensioned to fit within a working channel of the scoping device. Then, after insertion of the tubular member into the scoping device, the electrosurgical instrument may be inserted through the lumen of the tubular member, until the radiating tip of the electrosurgical instrument reaches a target treatment site within the patient.

The tubular member may include a thermally conductive material, e.g. the tubular member may be formed at least in part by a thermally conductive material. For example, the tubular member may be formed of a metal (e.g. aluminium, copper, brass, gold, diamond) or other thermally conductive material. In some cases, the tubular member may be formed by a hollow cylindrical tube made of thermally conductive material.

In some examples, the tubular member may include a thermally conductive dielectric (e.g. non-metallic) material. This may minimise interference of the tubular member with microwave energy radiated by a radiating tip of the electrosurgical instrument, and prevent back-propagation of microwave energy along the length of the tubular member. For example, the tubular member may be formed of a thermally conductive dielectric material. As an example, alumina (or aluminium oxide) may be a suitable thermally conductive dielectric material.

Herein, a thermally conductive material may be a material having a thermal conductivity of at least 10 W·m⁻¹·K⁻¹. Thus, the tubular member may include a material having a thermal conductivity of at least 10 W·m⁻¹·K⁻¹. In some embodiments, the thermal conductivity of the thermally conductive material may be at least 20 W·m⁻¹·K⁻¹, 40 W·m⁻¹·K⁻¹, 60 W·m⁻¹·K⁻¹, 80 W·m⁻¹·K⁻¹, 100 W·m⁻¹·K⁻¹, 120 W·m⁻¹·K⁻¹, 140 W·m⁻¹·K⁻¹, 160 W·m⁻¹·K⁻¹, 180 W·m⁻¹·K⁻¹ or 200 W·m⁻¹·K⁻¹. A higher thermal conductivity may enable more efficient removal of heat from the electrosurgical instrument inside the lumen of the tubular member. Examples of suitable thermally conductive materials include copper (thermal conductivity≈398 W·m⁻¹·K⁻¹), aluminium (thermal conductivity≈237 W·m⁻¹·K⁻¹), brass (thermal conductivity≈109 W·m⁻¹·K⁻¹), gold (thermal conductivity≈315 W·m⁻¹·K⁻¹), diamond (thermal conductivity≈1000-2200 W·m⁻¹·K⁻¹), alumina (thermal conductivity≈30 W·m⁻¹·K⁻¹). Other thermally conductive materials may also be used.

A rate of heat transfer through the tubular member may depend on a wall thickness of the tubular member. Accordingly, the wall thickness of the tubular member may be adjusted in order to achieve a desired rate of heat transfer through the tubular member.

The cooling assembly may include any suitable active and/or passive components configured to remove heat from the tubular member. This may serve to ensure that a temperature of the tubular member does not become too elevated, which could cause damage to surrounding tissue. This may also serve to remove heat from the electrosurgical instrument, in order to maintain the electrosurgical instrument at an acceptable temperature during use. Removing heat from the electrosurgical instrument may improve a performance of the electrosurgical instrument and avoid damage to the electrosurgical instrument caused by excessive heating. This may also enable the electrosurgical instrument to deliver higher power levels via the radiating tip which may, for example, enable larger target areas to be treated.

The cooling assembly may include a heat sink that is thermally coupled to the tubular member. The heat sink may thus serve to draw heat out of the tubular member, in order to reduce a temperature of the tubular member. The heat sink may be formed of a thermally conductive material, e.g. a metal such as copper, aluminium or brass. The heat sink may be in the form of a block of thermally conductive material that is thermally coupled to the tubular member. In some cases, the heat sink may include one or more fins. The one or more fins may serve to increase a surface area of the heat sink, which may facilitate cooling of the heat sink, e.g. when the heat sink is cooled with a fan and/or coolant.

The heat sink may be thermally coupled to the tubular member via any suitable link that enables heat to be transferred from the tubular member to the heat sink. For example, the heat sink may be thermally coupled to the tubular member via a wire or cable made of a thermally conductive material (e.g. metal). In some cases, the heat sink may be directly connected, e.g. in contact with, the tubular member.

The heat sink may have a greater heat capacity than the tubular member. This may cause heat to flow from the tubular member to the heat sink, so that heat is efficiently removed from the tubular member.

The heat sink may be disposed at or near a proximal end of the tubular member. In use, the proximal end of the tubular member may be located outside the patient, whilst a distal end of the tubular member may be located inside the patient's body. Placing the heat sink at or near a proximal end of the tubular member may thus ensure that heat is removed from the tubular element to a location that is outside the patient's body, as well as facilitate cooling the heat sink.

The heat sink may be disposed in a handle of the tubular member. This may facilitate integrating the heat sink into the introducer, and improve handling and maneuverability of the tubular member. The handle may be disposed at the proximal end of the tubular member. Thus, in use, a user may grip the tubular member by the handle, to facilitate positioning and insertion of the tubular member into the patient. The handle may be formed by the heat sink. Alternatively, the heat sink may be contained within the handle.

The heat sink may be thermally coupled to the tubular member via a heat pipe. This may provide a strong thermal link between the tubular member and the heat sink, to enable efficient removal of heat from the tubular member. The heat pipe may be a conventional heat pipe which is configured to transfer heat from the tubular member to the heat sink. A first end of the heat pipe may be connected to the tubular member, whilst a second end of the heat pipe may be connected to the heat sink. In some cases, the second end of the heat pipe may constitute the heat sink.

The cooling assembly may be configured to actively cool the heat sink. Actively cooling the heat sink may enable heat to be removed more efficiently from the heat sink, which may in turn enable more heat to be removed from the tubular member (and any electrosurgical instrument contained therein). Active cooling may refer to a type of cooling that requires electrical power. Active cooling may involve causing a fluid flow to come into contact with the heat sink, to remove heat from the heat sink.

The cooling assembly may include a fan configured to actively cool the heat sink. The fan may be arranged to produce an air flow which is directed at the heat sink, such that the heat sink is cooled by the air flow. This may enable heat to be efficiently removed from the heat sink.

The cooling assembly may be configured to actively cool the heat sink with a coolant fluid. The cooling assembly may be configured to cause a flow of coolant fluid to remove heat from the heat sink. Thus, the heat sink may act as a heat exchanger between the tubular member and the coolant fluid. In some cases, the coolant fluid may come into direct contact with, e.g. flow over and/or through, the heat sink. In other cases, the coolant fluid may not come into direct contact with the heat sink, but may flow through a tube or conduit that is in thermal contact with the heat sink.

The coolant fluid may be a liquid or a gas. For example, suitable coolant fluids may include water, liquid or gas nitrogen, and liquid or gas helium.

The cooling assembly may include a coolant fluid source, which is arranged to produce a flow of coolant fluid. The coolant source may be connected to the heat sink via one or more conduits, so that coolant fluid may flow from the coolant fluid source to the heat sink. As an example, the coolant fluid source may be in the form of a pressurised container that contains the coolant fluid. As another example, the coolant fluid source may be in the form of a coolant fluid container having a pump or other suitable mechanism for causing the coolant fluid to flow to the heat sink.

The heat sink may include one or more channels formed therein through which the coolant fluid may flow. This may promote heat exchange between the coolant fluid and the heat sink.

The cooling assembly may include a heat exchanger configured to actively cool the heat sink. The heat sink may be thermally coupled to the heat exchanger, such that heat is removed from the heat sink by the heat exchanger. For example, the heat exchanger may include a hot end and a cold end, with the heat sink being thermally coupled to the cold end of the heat exchanger.

Alternatively, the heat sink may be passively cooled. For example, the heat sink may be left in air so that it is cooled by the air. In some cases, the heat sink may be brought into contact with a coolant, e.g. the heat sink may be placed in a vessel containing a coolant. Suitable coolants may include, for example, water, liquid nitrogen, or liquid helium.

The cooling assembly may include a heat pump configured to remove heat from the tubular member. For example, the heat pump may be a thermoelectric heat pump such as a Peltier cooler. The heat pump may be mounted directly on a surface of the tubular member, e.g. near a proximal end of the tubular member. In cases where the cooling assembly includes a heat sink, the heat pump may be configured to remove heat from the heat sink, e.g. the heat pump may be mounted on a surface of the heat sink.

The tubular member may include one or more channels defined on or in a sidewall of the tubular member, and the cooling assembly may be configured to circulate a coolant fluid through the one or more channels to remove heat from the tubular member. By circulating coolant fluid through the one or more channels, heat may be removed from the tubular member. The one or more channels may extend along a length of the tubular member. This may enable heat to be removed along the length of the tubular member, which may result in a substantially uniform temperature along the length of the tubular member. For example, the one or more channels may extend from a proximal end of the tubular member to a position at or near a distal end of the tubular member.

The cooling assembly may include a coolant fluid source configured to circulate the coolant fluid through the one or more channels. The coolant fluid may be a liquid or a gas. For example, suitable coolant fluids may include water, liquid or gas nitrogen, and liquid or gas helium.

The one or more channels may include a first channel (“in channel”) via which coolant fluid may be introduced into the tubular member, and a second channel (“out channel”) through which the coolant fluid may flow out of the tubular member. The first channel and second channel may be connected via a connecting channel, e.g. located near the distal end of the tubular member. An inlet of the first channel may be connected to the coolant fluid source at its proximal end to receive coolant fluid from the coolant fluid source. An outlet of the second channel may be connected as appropriate for collecting or recirculating exhaust coolant fluid flowing out of the second channel.

The tubular member may include one or more contact elements disposed within the lumen and arranged to press against an outer surface of the electrosurgical instrument when the electrosurgical instrument is inserted through the lumen. The one or more contact elements may serve to provide a thermal link between the tubular member and the electrosurgical instrument when it is received in the lumen. The one or more contact elements may be made of a thermally conductive material, e.g. metal.

The one or more contact elements may include a resilient material. This may serve to ensure that contact is maintained with the electrosurgical instrument, whilst facilitating insertion of the electrosurgical instrument through the lumen. For example, each of the one or more contact elements may include a spring or spring-like element that is configured to press the contact element against the outer surface of the electrosurgical element when the electrosurgical instrument is inserted through the lumen.

The tubular member may include a distal end formed of a dielectric material. This may minimise interference of the distal end of the tubular member with microwave energy radiated by the radiating tip of the electrosurgical instrument, and reduce back-propagation of microwave energy along the length of the tubular member. For example, the distal end of the tubular member may be made of a ceramic material (e.g. Zirconia), or a polymer material (e.g. Polyether ether ketone, or PEEK).

The tubular member may include a pointed distal end. A pointed distal end may enable the tubular member to pierce skin, to facilitate percutaneous insertion of the tubular member into a patient. In this manner, the tubular member may be used to pierce a patient's skin, and introduce the electrosurgical instrument percutaneously into a patient. Where the tubular member includes a distal end formed of a dielectric material, the pointed distal end may be formed by the dielectric material.

The pointed distal end may be formed integrally with the rest of the tubular member, e.g. where the distal end is formed of the same material as the rest of the tubular member. Alternatively, where the distal end is formed of a different material compared to the rest of the tubular member, the distal end may be secured at the distal end of the tubular member via any suitable means (e.g. via an adhesive or mechanical fixing mechanism).

The tubular member may include an outer layer formed of a biocompatible material. For example, the tubular member may include an inner layer formed of a thermally conductive material that is coated with or covered by the biocompatible material. The thermally conductive material may be a metal (e.g. aluminium, copper, brass). The biocompatible material may be a metal (e.g. gold or silver), or the biocompatible material may be a polymer material (e.g. polytetrafluoroethylene—PTFE). The biocompatible material may serve to protect the tubular member. In some cases, the biocompatible material may be a non-stick material (e.g. PTFE), to facilitate insertion of the tubular member into the patient.

The tubular member may include an inner layer formed of a thermally conductive material, and an outer layer formed of a thermally insulating material. In this manner, the thermally conductive inner layer may absorb and conduct heat from the electrosurgical instrument when the electrosurgical instrument is received in the lumen. The thermally insulating outer layer may act as a thermal barrier against heat generated by the electrosurgical instrument, in order to minimise heating of surrounding tissue. The inner layer and outer layer may be formed of different materials which are secured together.

A thermal conductivity of the thermally conductive material may be greater than a thermal conductivity of the thermally insulating material. Any of the thermally conductive materials discussed above may be used as the thermally conductive material for the inner layer (e.g. copper, brass, aluminium, etc.). Examples of suitable thermally insulating materials include polystyrene (thermal conductivity≈0.0081-0.026 W·m⁻¹·K⁻¹), polyurethane (thermal conductivity≈0.032-0.05 W·m⁻¹·K⁻¹), glass (thermal conductivity≈0.18-0.96 W·m⁻¹·K⁻¹), mica (thermal conductivity≈0.71 W·m⁻¹·K⁻¹), PTFE (thermal conductivity≈0.25 W·m⁻¹·K⁻¹). However, other thermally insulating materials may also be used. Generally speaking, a thermally insulating material may include a material having a thermal conductivity below 10 W·m⁻¹·K⁻¹. Preferably, a thermally insulating material may include a material having a thermal conductivity below 1 W·m⁻¹·K⁻¹.

The inner layer and the outer layer may be formed as concentric tubes. The concentric tubes may be secured together, e.g. via a suitable adhesive or epoxy.

In some cases, the inner layer may be formed as a coating on an inner surface of the outer layer. For example, the outer layer may be formed as a hollow tube of thermally insulating material (e.g. mica). The inner layer may then be formed by coating an inner surface of the hollow tube with the thermally conductive material (e.g. gold).

A thickness of the outer layer may be greater than a thickness of the inner layer. This may reduce leakage of heat from the electrosurgical instrument into surrounding tissue. For example, a thickness of the inner layer may be between 15%-25% of a thickness of the outer layer.

Where the tubular member includes an outer layer formed of a biocompatible material, the biocompatible outer layer may be made of a thermally insulating material. For example, the outer layer may be formed of PTFE.

Where the tubular member includes the above-mentioned inner layer and outer layer, the cooling assembly may be configured to remove heat from one or both of the inner layer and the outer layer of the tubular member. In some cases, the cooling assembly may be directly coupled to the inner layer, in order to remove heat more efficiently from the inner layer. For example, the outer layer may include one or more holes via which the cooling assembly is thermally coupled to the inner layer.

Where the tubular member includes the above-mentioned inner layer and outer layer, and where the tubular member includes one or more channels as described above, the one or more channels may be defined in the inner and/or outer layer of the tubular member. In one example, the one or more channels may include a first channel (“in channel”) via which coolant fluid is introduced into the tubular member, the first channel being formed in the inner layer of the tubular member. The one or more channels may also include a second channel (“out channel”) through which coolant fluid may flow out of the tubular member, the second channel being formed in the outer layer of the tubular member. In this manner, the coolant fluid may first pass through the inner layer, and then through the outer layer, such that heat may be removed more efficiently from the inner layer.

In some embodiments, a proximal portion of the tubular member may be flexible, and a distal portion of the tubular member may be rigid. A stiffness of the distal portion of the tubular member may be greater than a stiffness of the proximal portion of the tubular member. In use, the rigid distal portion of the tubular member may be inserted into to the patient, whilst the flexible proximal of the tubular member may be disposed outside the patient. Making the distal portion of the tubular member rigid may facilitate insertion of the distal portion into the patient. Making the proximal portion flexible may facilitate handling of the introducer. In particular, the flexible portion of the tubular member may be beneficial where the electrosurgical instrument includes a long cable or transmission line, as the flexible portion of the tubular member may enable the cable of the electrosurgical instrument to flex or bend, which may facilitate connecting the electrosurgical instrument to an electrosurgical generator. A length of the proximal portion may be greater than a length of the distal portion, e.g. to facilitate receiving a cable of the electrosurgical instrument in the proximal portion.

The distal portion of the tubular member may be include a rigid (or stiff) thermally conductive material. For example, the distal portion of the tubular member may be include a rigid metal tube.

The proximal portion of the tubular member may include a flexible thermally conductive material. Herein a flexible material may refer to a material that is bendable or supple. For example, the proximal portion of the tubular member may be made of braided material, e.g. a metallic braid, to provide flexibility to the proximal portion.

A length of the tubular member may be 30 cm or greater. Such a length may facilitate percutaneous insertion of the tubular into the body of a patient, as well as ensure that the patient's body is adequately shielded from heat generated by the electrosurgical instrument.

The introducer of the first aspect of the invention may form part of an electrosurgical system. Thus, according to a second aspect of the invention, there is provided an electrosurgical system comprising: an electrosurgical instrument comprising: a transmission line for conveying microwave and/or radiofrequency electromagnetic (EM) energy; and a radiating tip mounted at a distal end of the transmission line configured to receive and deliver the microwave and/or radiofrequency EM energy to biological tissue; and an introducer according to the first aspect of the invention, wherein the electrosurgical instrument is insertable through the lumen of the tubular member. Any of the features of the introducer discussed above in relation to the first aspect of the invention may be shared with the second aspect of the invention.

The electrosurgical instrument may be configured to deliver microwave and/or radiofrequency EM energy to biological tissue. Microwave and/or radiofrequency EM energy may be conveyed along the transmission line to the radiating tip, which is configured to deliver the EM energy to biological tissue. The radiating tip may include one or more electrodes which are arranged to deliver the EM energy to the biological tissue.

The transmission line may be any suitable cable for conveying microwave and/or radiofrequency EM energy. For example the transmission line may be a flexible coaxial cable. A distal end of the transmission line may be electrically connected to the radiating tip, to transfer EM energy from the transmission line to the radiating tip.

The electrosurgical instrument may be dimensioned such that it is insertable through the lumen of the tubular member. For example, an outer diameter of the electrosurgical instrument may be less than, or equal to, a diameter of the lumen of the tubular member. In use, a portion of the transmission line may be received in the lumen of the tubular member, whilst the radiating tip may protrude from a distal end of the tubular member. In this manner, the radiating tip may deliver EM energy to target tissue, whilst the tubular member may act to protect the patient's body from heat generated in the transmission line.

The system may further comprise an electrosurgical generator configured to generate the microwave and/or radiofrequency EM energy. The electrosurgical generator may be electrically connected to a proximal end of the transmission line, to deliver the EM energy to the transmission line.

According to a third aspect of the invention, there is provided a method of introducing an electrosurgical instrument into a body of a patient, the method comprising: inserting a tubular member of an introducer into the body of the patient; inserting an electrosurgical instrument through a lumen of the tubular member, such that a radiating tip of the electrosurgical instrument protrudes beyond a distal end of the tubular member; and removing heat from the tubular member using a cooling assembly of the introducer.

The introducer may be an introducer according to the first aspect of the invention. The introducer and electrosurgical instrument may be part of an electrosurgical system according to the second aspect of the invention.

The tubular member may be inserted percutaneously into the body of the patient. Alternatively, the tubular member may be inserted into the body of the patient via a surgical scoping device, e.g. via a laparoscope.

The electrosurgical instrument may be inserted through the lumen of the tubular member until the radiating tip of the electrosurgical instrument protrudes beyond the distal end of the tubular member. In this manner, the radiating tip may be exposed, such that it may come into contact with target tissue.

Heat may then be removed from the tubular member using a cooling assembly of the introducer. This may include passively and/or actively removing heat from the tubular member, depending of the configuration of the cooling assembly used. Where the cooling assembly includes an active component (e.g. a fan), removing heat from the tubular assembly may include activating the active component of the cooling assembly.

Herein, the term “inner” may refer to a position that is radially closer to the centre (e.g. axis) of the tubular member and/or electrosurgical instrument. The term “outer” may refer to a position that is radially further from the centre (axis) of the tubular member and/or electrosurgical instrument.

The term “conductive” is used herein to mean electrically conductive, unless the context dictates otherwise.

Herein, the terms “proximal” and “distal” refer to the ends of the tubular member or electrosurgical instrument. In use, the proximal end is closer to a generator for providing the RF and/or microwave energy, whereas the distal end is further from the generator. In particular, the proximal ends of the tubular member and electrosurgical instrument may be disposed outside the patient's body, whilst the distal ends of the tubular member and electrosurgical instrument may be disposed inside the patient's body, e.g. in the vicinity of target tissue.

In this specification “microwave” may be used broadly to indicate a frequency range of 400 MHz to 100 GHz, but preferably the range 1 GHz to 60 GHz. Preferred spot frequencies for microwave EM energy include: 915 MHz, 2.45 GHz, 3.3 GHz, 5.8 GHz, 10 GHz, 14.5 GHz and 24 GHz. 5.8 GHz may be preferred. The device may deliver energy at more than one of these microwave frequencies.

The term “radiofrequency” or “RF” may be used to indicate a frequency between 300 kHz and 400 MHz.

### BRIEF DESCRIPTION OF THE DRAWINGS

Examples of the invention are discussed below with reference to the accompanying drawings, in which:

FIG. 1 is a schematic diagram of an introducer according to an embodiment of the invention;

FIG. 2 is a schematic diagram of an introducer according to an embodiment of the invention;

FIG. 3 is a schematic diagram of an introducer according to an embodiment of the invention;

FIG. 4 is a cross-sectional diagram of a tubular member that may form part of an introducer according to an embodiment of the invention;

FIG. 5 is a cross-sectional diagram of a tubular member that may form part of an introducer according to an embodiment of the invention;

FIG. 6 is a schematic diagram of an introducer according to an embodiment of the invention; and

FIG. 7 is a schematic diagram of an introducer according to an embodiment of the invention.

### DETAILED DESCRIPTION; FURTHER OPTIONAL FEATURES

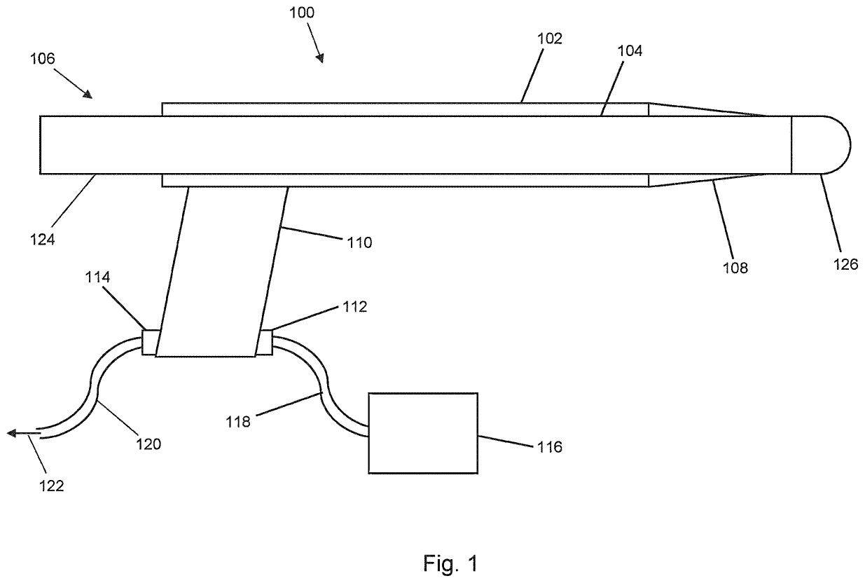

FIG. 1 shows a schematic diagram of an introducer 100 according to an embodiment of the invention. The introducer 100 includes a tubular member 102 that defines a lumen 104 through which an electrosurgical instrument 106 is insertable.

The tubular member 102 is formed by a hollow cylindrical tube of thermally conductive material. For example, the tubular member may be made of copper, aluminium or brass. The tubular member may also be coated with a non-stick biocompatible material, such as PTFE, in order to facilitate percutaneous insertion of the tubular member into a body of a patient.

The lumen 104 in the tubular member 102 is dimensioned to receive the electrosurgical instrument 106. In particular, the lumen 104 is dimensioned such that when the electrosurgical instrument 106 is inserted through the lumen 104, an outer surface of the electrosurgical instrument 106 is in contact with a wall of the lumen 104. For example, a cross-sectional area of the lumen 104 may substantially match a cross-sectional area of the electrosurgical instrument 106. In this manner, the electrosurgical instrument 106 may be thermally coupled to the tubular member 102, such that heat may flow from the electrosurgical instrument 106 to the tubular member 102. The lumen 104 and electrosurgical instrument 106 may have substantially circular cross-sectional areas.

The tubular member 102 includes a pointed distal end 108 which is formed of a dielectric material, e.g. PEEK. The pointed distal end 108 may be secured to the rest of the tubular member 102 via a suitable adhesive. The pointed distal end 108 may facilitate piercing of a patient's skin, to enable the tubular member to be inserted percutaneously into the patient.

The tubular member 102 further includes a heat sink 110 which is attached at a proximal end of the tubular member 102. The heat sink 110 is thermally coupled to the tubular member 102, such that heat may flow from the tubular member 102 to the heat sink 110. The heat sink 110 is in the form of a block of thermally conductive material (e.g. copper, aluminium, brass). The heat sink 110 is arranged such that it has a greater heat capacity than the tubular member 102, so that the heat may preferentially flow from the tubular member 102 to the heat sink 110. In this manner, the heat sink 110 may efficiently remove heat from the tubular member 102.

As the heat sink 110 is attached to a proximal end of the tubular member 102, it may also serve as a handle for the tubular member 102. Thus, a user may grip the tubular member via the heat sink 110, which may facilitate manipulation of the tubular member 102. The heat sink 110 may be ergonomically shaped, to facilitate gripping of the heat sink 110. Additionally, the heat sink 110 may be covered with a grip (e.g. anti-slip) material such as rubber or the like, to facilitate gripping of the heat sink 110.

The heat sink 110 includes a passageway defined therein (not shown) through which a coolant fluid may flow, in order to remove heat from the heat sink 110. The passageway defined in the heat sink 110 extends between an inlet 112 and an outlet 114 of the heat sink 110. The passageway in the heat sink 110 may define a convoluted path, in order to maximise heat removal from the heat sink 110 by coolant fluid flowing through the passageway.

The introducer 100 further includes a coolant fluid source 116, which is configured to cause coolant fluid to flow through the passageway in the heat sink 110. The coolant fluid source 116 is coupled to the inlet 112 of the heat sink 110 via a first conduit 118 (or tube), such that coolant fluid may flow from the coolant fluid source 116 into the inlet 112 of the heat sink 110. A second conduit 120 is connected to the outlet 114 of the heat sink 110, so that the coolant fluid may flow out of the heat sink 110 via the second conduit 120, as illustrated by arrow 122. Coolant fluid exiting via the second conduit 120 may, for example, be captured in a reservoir for exhaust coolant fluid. As another example, coolant fluid exiting via the second conduit 120 may be recirculated to the coolant fluid source 116 so that it may be reused.

The coolant fluid source 116 includes a reservoir (or tank) containing coolant fluid. The coolant fluid may include a gas or a liquid. Suitable coolant fluids may include, for example, water, liquid or gas nitrogen, liquid or gas helium. The coolant fluid source 116 is configured to cause coolant fluid contained in the reservoir to flow through the passageway in the heat sink 110. For example, the coolant fluid in the reservoir may be pressurised, and the coolant fluid source 116 may include a valve for controlling flow of the coolant fluid out of the reservoir and into the first conduit 118. As another example, the coolant fluid source 116 may include a pump or other mechanism for causing coolant fluid to flow from the reservoir into the first conduit 118. Together, the heat sink 110, coolant fluid source 116 and conduits 118, 120 may form a cooling assembly of the introducer 100.

In use, the tubular member 102 may initially be inserted into a body of a patient. The pointed distal end 108 may be used to pierce the patient's skin. The tubular member 102 may be inserted to a desired depth, such that the pointed distal end 108 of the tubular member 102 is in the vicinity of target biological tissue in the patient. Then, the electrosurgical instrument 106 may be inserted through the lumen 104 in the tubular member 102.

The electrosurgical instrument 106 is configured to deliver RF and/or microwave energy to biological tissue. The electrosurgical instrument includes a transmission line 124, and a radiating tip 126 disposed at a distal end of the transmission line 124. The transmission line 124 is configured to convey the RF and/or microwave energy from an electrosurgical generator connected at a proximal end of the transmission line 124; for example, the transmission line 124 may be a suitable coaxial cable. The radiating tip 126 is electrically connected to the transmission line 124 to receive the RF and/or microwave energy, and deliver the RF and/or microwave energy to target tissue. The radiating tip may include one or more electrodes (not shown) for delivering the RF and/or microwave energy to biological tissue. The one or more electrodes may be arranged depending on the type of energy to be delivered, and the type of electrosurgery to be performed.

The electrosurgical instrument 106 may be inserted through the lumen 104 of the tubular member 102 until the radiating tip 126 protrudes beyond the pointed distal end 108 of the tubular member 102, as shown in FIG. 1. In this manner, the radiating tip 126 may be exposed so that it may deliver the RF and/or microwave to target tissue. In this configuration, a portion of the transmission line 124 is received within the lumen 104 of the tubular member 102. The portion of the transmission line 124 in the lumen 104 is in direct contact with the tubular member 102, such that they are in thermal contact with one another.

The cooling assembly of the introducer 100 may be activated, e.g. by activating the coolant fluid source 116 so that coolant fluid is caused to flow through passageway in the heat sink 110. This serves to remove heat from the heat sink 110.

As RF and/or microwave energy is conveyed along the transmission line 124 and delivered to target tissue via the radiating tip 126, the electrosurgical instrument 106 may heat up, e.g. due to losses in the transmission line 124. As a result, heat may flow from the electrosurgical instrument 106 into the tubular member 102, which may cause the tubular member 102 to heat up. Heat may then flow from the tubular member 102 into the heat sink 110. The coolant fluid source 116 may be activated in order to cause coolant fluid to flow through the passageway in the heat sink 110, in order to remove heat from the heat sink 110. As the coolant fluid flows through the passageway in the heat sink 110, it may absorb heat from the heat sink 110. In this manner, the heat sink 110 may be effectively cooled, so that it can continue to absorb heat from the tubular member 102. As a result, the tubular member 102 may be maintained at a relatively low temperature, which may avoid damage to surrounding tissue. Additionally, heat generated by the electrosurgical instrument 106 may be effectively be removed via the tubular member 102 and heat sink 110, which may serve to keep the electrosurgical instrument 106 at a suitable working temperature. A flow rate of the coolant fluid through the passageway in the heat sink 110 may be controlled (e.g. by controlling the coolant fluid source 116), in order to control cooling of the heat sink 110, e.g. so the heat sink 110 (and hence also the tubular member 102) may be maintained at a desired temperature.

Together, the electrosurgical instrument 106 and introducer 100 may form part of an electrosurgical system that is an embodiment of the invention. Such an electrosurgical system may further include an electrosurgical generator which is connected (or connectable) at a proximal end of the transmission line 124, and configured to deliver RF and/or microwave energy to the transmission line 124.

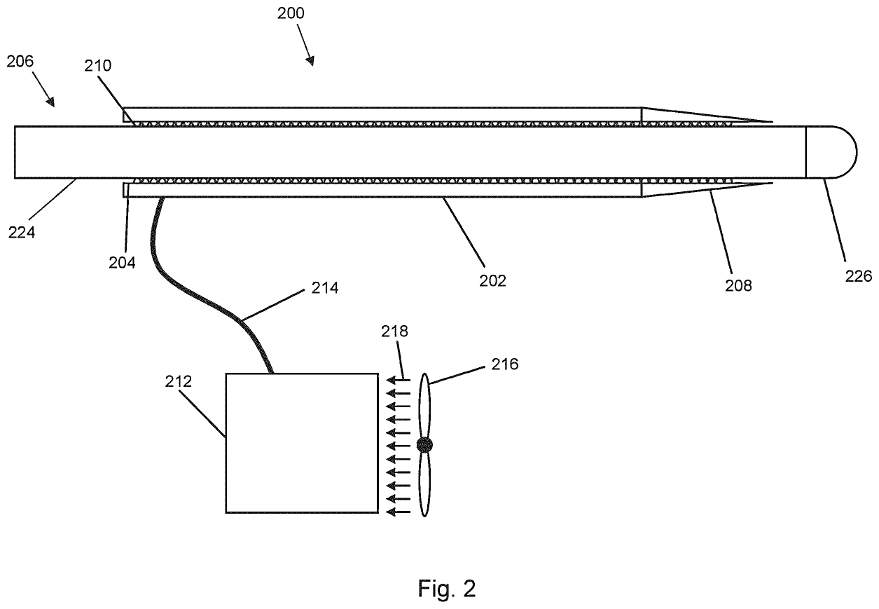

FIG. 2 shows a schematic diagram of an introducer 200 according to another embodiment of the invention. The introducer 200 includes a tubular member 202 that defines a lumen 204 through which an electrosurgical instrument 206 is insertable. The electrosurgical instrument 206 includes a transmission line 224 and radiating tip 226, and is similar in configuration to electrosurgical instrument 106 discussed above.

Similarly to tubular member 102 discussed above, the tubular member 202 is formed by a hollow cylindrical tube of thermally conductive material, which may be coated with a non-stick biocompatible material. A pointed distal end 208 made of a dielectric material (e.g. PEEK) is disposed at a distal end of the tubular member 202.

The lumen 204 is dimensioned to receive the electrosurgical instrument 206. For example, a cross-sectional area of the lumen 204 may be slightly larger than a cross-sectional area of the electrosurgical instrument 206. A plurality of contact elements 210 are disposed on a wall of the lumen 204, and arranged to press against an outer surface of the electrosurgical instrument 206 when the electrosurgical instrument 206 is inserted through the lumen 204. The contact elements 210 are made of a thermally conductive material, and serve to provide a thermal link between the electrosurgical instrument 206 and the tubular member 202. The contact elements 210 may be made of a resilient (e.g. flexible) material. This may facilitate inserting the electrosurgical instrument 206 into the lumen 204, whilst ensuring that thermal contact is maintained between the electrosurgical instrument 206 and the tubular member 202. Each of the embodiments illustrated in FIGS. 1, 3, 4a, 4b and 5 may be modified to include contact elements similar to contact elements 210 in the lumen of its tubular member.

The introducer 200 further includes a heat sink 212. The heat sink 212 is thermally coupled to the tubular member 202 via a thermal link in the form of a heat pipe 214. The heat sink 212 is in the form of a block of thermally conductive material (e.g. copper, aluminium, brass), and has a heat capacity that is larger than a heat capacity of the tubular member 202. The heat sink 212 includes a series of fins (not shown) arranged on its surface, in order to increase a surface area of the heat sink 212. The heat pipe 214 may be any suitable conventional heat pipe, and acts to conduct heat from the tubular member 202 to the heat sink 212. The heat pipe 214 is connected to the tubular member 202 near a proximal end of the tubular member 202.

The introducer 200 further includes a fan 216 which is configured to actively cool the heat sink 212. In particular, the fan 216 is configured to blow air onto the heat sink 212, as illustrated by arrows 218, in order to cool the heat sink 212. The fan 216 may be any suitable conventional fan, e.g. an electric fan. The fan 216 may be powered by an external power source (not shown). Together, the heat pipe 214, heat sink 212 and fan 216 may form a cooling assembly of the introducer 200.

In the configuration shown in FIG. 2, the electrosurgical instrument 206 is inserted into the lumen 204 of the tubular member 202, such that a portion of the transmission line 224 is disposed within the lumen 204, and the radiating tip 226 protrudes beyond the pointed distal end 208 of the tubular member 202. During use of the electrosurgical instrument 206, heat generated by the electrosurgical instrument 206 may be transferred to the tubular member 202, via the plurality of contact elements 210. Heat may then flow from the tubular member 202 to the heat sink 212 via the heat pipe 214. The fan 216 may be activated in order to blow air across the heat sink 212, to remove heat from the heat sink 212, so that the heat sink can effectively absorb heat from the tubular member 202. In this manner, the tubular member 202 may be maintained at a relatively low temperature, which may avoid damage to surrounding tissue. Additionally, heat generated by the electrosurgical instrument 206 may be effectively be removed via the tubular member 202 and heat sink 212, which may serve to keep the electrosurgical instrument 206 at a suitable working temperature.

In an alternative embodiment (not shown), instead of actively cooling the heat sink with the fan 216, the heat sink 212 may be passively cooled using a coolant fluid. For example, the heat sink 212 may be submerged in a vessel containing a coolant fluid (e.g. water, liquid nitrogen, or liquid helium). In this manner, the coolant fluid may cool the heat sink 212, so that it may efficiently absorb heat from the tubular member 202.

Together, the electrosurgical instrument 206 and introducer 200 may form part of an electrosurgical system that is an embodiment of the invention. Such an electrosurgical system may further include an electrosurgical generator which is connected (or connectable) at a proximal end of the transmission line 224, and configured to deliver RF and/or microwave energy to the transmission line 224.

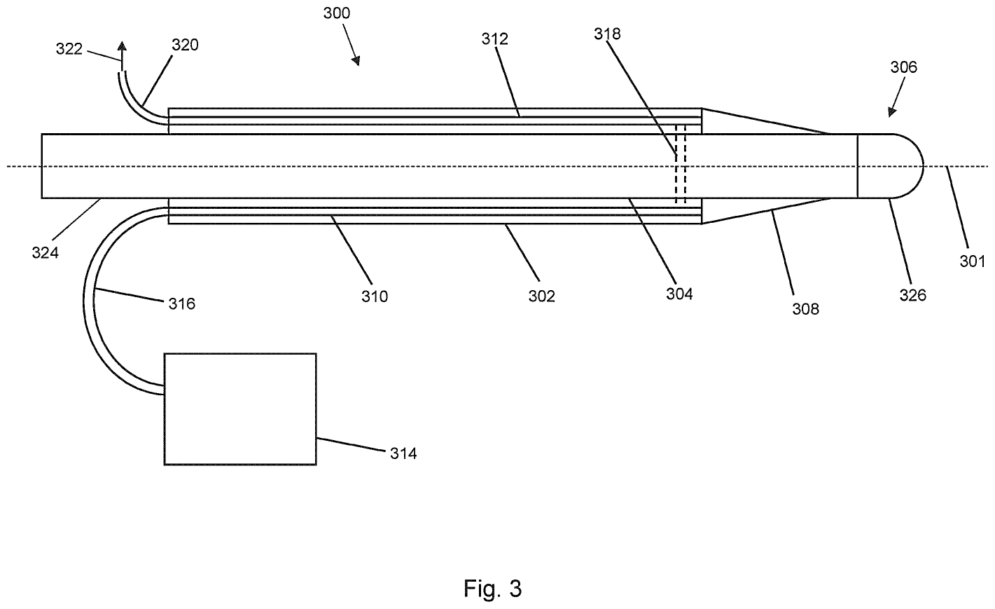

FIG. 3 shows a schematic diagram of an introducer 300 according to another embodiment of the invention. The introducer 300 includes a tubular member 302 that defines a lumen 304 through which an electrosurgical instrument 306 is insertable. The electrosurgical instrument 306 includes a transmission line 324 and radiating tip 326, and is similar in configuration to electrosurgical instrument 106 discussed above.

Similarly to tubular members 202 and 102 discussed above, the tubular member 302 is formed by a hollow cylindrical tube of thermally conductive material, which may be coated with a non-stick biocompatible material. A pointed distal end 308 made of a dielectric material (e.g. PEEK) is disposed at a distal end of the tubular member 302.

The lumen 304 in the tubular member 302 is dimensioned to receive the electrosurgical instrument 306. In particular, the lumen 304 is dimensioned such that when the electrosurgical instrument 306 is inserted through the lumen 304, an outer surface of the electrosurgical instrument 306 is in contact with a wall of the lumen 304. For example, a cross-sectional area of the lumen 304 may substantially match a cross-sectional area of the electrosurgical instrument 306.

The tubular member 304 includes a first channel 310 and a second channel 312 defined in a sidewall of the tubular member 302. The first channel 310 and second channel 312 extend from a proximal end of the tubular member 302 towards a distal end of the tubular member 302. A proximal end of the first channel 310 is connected to a coolant fluid source 314 via a first conduit 316, so that coolant fluid may flow from the coolant fluid source 314 into the first channel 310.

The first channel 310 and second channel 312 are connected together near the distal end of the tubular member 302 via a connecting channel 318 (shown by the dashed lines in FIG. 3). The connecting channel 318 is defined within the sidewall of the tubular member 302, and extends between the first channel 310 and the second channel 312. In this manner, coolant fluid may flow from the first channel 310 into the second channel 312 via the connecting channel 318. A second conduit 320 is connected to a proximal end of the second channel 312, such that coolant fluid may flow out from the second channel into the second conduit 320. In this manner, the first conduit 316, first channel 310, connecting channel 318, second channel 312 and second conduit 320 form a flow path along which coolant fluid may flow.

The coolant fluid source 314 is configured to circulate coolant fluid along this flow path, so that coolant fluid flows through the first and second channels 310, 312 in the tubular member 302. The coolant fluid source 314 may have a similar configuration to coolant fluid source 116 described above. For example, the coolant fluid source 314 may include a reservoir containing a coolant fluid, and it may be configured to cause coolant fluid to flow from the reservoir into first conduit 316. This may be achieved by pressurising the reservoir, and controlling flow of coolant fluid out of the reservoir via a valve, or by providing a pump or other mechanism for causing the coolant fluid to flow out of the reservoir. The coolant fluid may include a gas or a liquid. Suitable coolant fluids may include, for example, water, liquid or gas nitrogen, liquid or gas helium.

In the configuration shown in FIG. 3, the electrosurgical instrument 306 is inserted into the lumen 304 of the tubular member 302, such that a portion of the transmission line 324 is disposed within the lumen 304, and the radiating tip 326 protrudes beyond the pointed distal end 308 of the tubular member 302. During use of the electrosurgical instrument 306, heat generated by the electrosurgical instrument 306 may be transferred to the tubular member 302. The coolant fluid source 314 may be activated, to cause coolant fluid to flow along the flow path, such that it flows along the first and second channels 310, 312. As the coolant fluid flows along the first and second channels 310, 312, it may absorb heat from the tubular member 302, such that the tubular member 302 is cooled. In this manner, the tubular member 302 may be maintained at a safe temperature, to avoid damage to surrounding tissue.

During operation of the electrosurgical instrument 306, the coolant fluid source 314 may be configured to continuously flow coolant fluid along the flow path, such that heat is continuously removed from the tubular member 302. A flow rate of the coolant fluid along the flow path may be adjusted (e.g. by controlling the coolant fluid source 314), in order to control cooling of the tubular member 302, e.g. so that the tubular member 302 may be maintained at a desired temperature.

Exhaust coolant fluid may then exit via the second conduit 320, as illustrated by arrow 322. Coolant fluid exiting via the second conduit 320 may, for example, be captured in a reservoir for exhaust coolant fluid. As another example, coolant fluid exiting via the second conduit 320 may be recirculated to the coolant fluid source 314 so that it may be reused.

Together, the electrosurgical instrument 306 and introducer 300 may form part of an electrosurgical system that is an embodiment of the invention. Such an electrosurgical system may further include an electrosurgical generator which is connected (or connectable) at a proximal end of the transmission line 324, and configured to deliver RF and/or microwave energy to the transmission line 324.

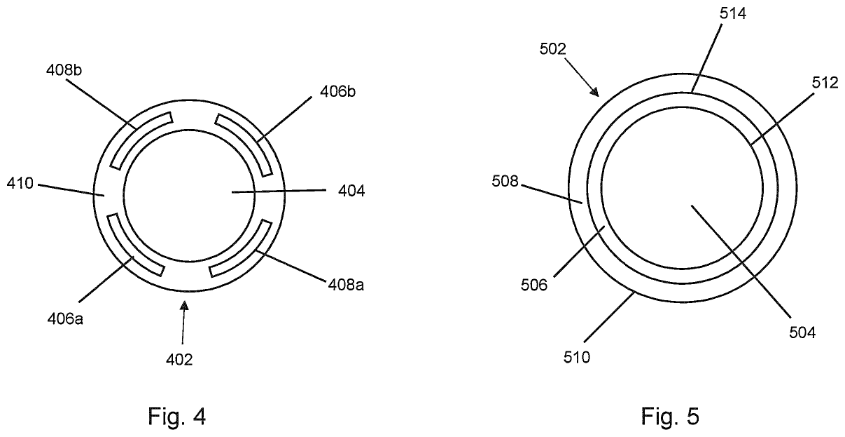

FIGS. 4 and 5 illustrate tubular members including different configurations of channels which may be used for circulating a coolant fluid. FIG. 4 shows a cross-sectional view of a tubular member 402 that may form part of an introducer according to an embodiment of the invention. The tubular member 402 is formed by a hollow cylindrical tube of thermally conductive material. The tubular member 402 defines a lumen 404 which extends along a length of the tubular member 402, and through which an electrosurgical instrument may be inserted. A first pair of channels 406a, 406b and a second pair of channels 408a, 408b are defined in a sidewall 410 of the tubular member 402.

Similarly to the first channel 310 and second channel 312 discussed above, channels 406a, 406b, 408a and 408b extend along a length of the tubular member 402, i.e. from a proximal end of the tubular member 402 to a position near a distal end of the tubular member 402. The first pair of channels 406a, 406b may be fluidly connected to the second pair of channels 408a, 408b via a set of connecting channels (not shown) which are defined in the sidewall 410 of the tubular member 402 near a distal end of the tubular member 402. For example, a first connecting channel may be arranged to connect channel 406a to channel 408a, and a second connecting channel may be arranged to connect channel 406b to channel 408b.

The first pair of channels 406a, 406b may be used as “in channels”, via which coolant fluid is introduced into the tubular member 402, whilst the second pair of channels 408a, 408b may be used as “out channels”, via which the coolant fluid may flow out of the tubular member 402. For example, a coolant fluid source (e.g. similar to coolant fluid source 314) may be connected via a pair of conduits to proximal ends of the first pair of channels 406a, 406b, such the coolant fluid may flow from the coolant fluid source into the first pair of channels 406a, 406b. The coolant fluid may then flow along the first pair of channels 406a, 406b towards the distal end of the tubular member 402. At the distal end of the tubular member 402, the coolant fluid may pass into the second pair of channels 408a, 408b via the set of connecting channels, and then flow back towards the proximal end of the tubular member 402, where the coolant fluid may exit the tubular member 402. Similarly to the discussion above of first and second channels 310, 312, as the coolant fluid flows through the channels 406a, 406b, 408a and 408b, heat may be removed from the tubular member 402 (and hence from an electrosurgical instrument received in the lumen 404).

The channels 406a, 406b of the first pair are arranged at diametrically opposite positions in the sidewall 410 relative to a longitudinal axis of the tubular member 402. Similarly, the channels 408a, 408b of the second pair are arranged at diametrically opposite positions in the sidewall 410 relative to the longitudinal axis of the tubular member 402. Such a configuration may result in a more uniform heat removal around a circumference of the tubular member.

FIG. 5 shows a cross-sectional view of a tubular member 502 that may form part of an introducer according to an embodiment of the invention. The tubular member 502 is formed by a hollow cylindrical tube of thermally conductive material. The tubular member 502 defines a lumen 504 which extends along a length of the tubular member 502, and through which an electrosurgical instrument may be inserted.

A pair of concentric channels are defined within a sidewall 510 of the tubular member 502. In particular, the tubular member 502 includes a first annular channel 506 which is disposed concentrically around the lumen 504, and a second annular channel 508 which is disposed concentrically around the first annular channel 506. The first annular channel 506 is separated from the lumen 504 by an inner wall 512, which also serves to define the lumen 504. The first annular channel 506 is separated from the second annular channel 508 by a separation wall 514. The first annular channel 506 and second annular channel 508 extend along a length of the tubular member 502, i.e. from a proximal end of the tubular member 502 to a position near a distal end of the tubular member 502. The first annular channel 506 and second annular channel 508 are connected together near the distal end of the tubular member, e.g. via one or more connecting passageways formed in the separation wall 514. In this manner, the first annular channel 506 and second annular channel 508 define a flow path along which coolant fluid may be made to flow.

For example, the first annular channel 506 may include an inlet (not shown) disposed near a proximal end of the tubular member 502. A coolant fluid source (e.g. similar to coolant fluid source 314) may be connected to the inlet of the first annular channel 506, so that coolant fluid may flow from the coolant fluid source into the first annular channel 506. The coolant fluid may then flow along the first annular channel 506 towards the distal end of the tubular member 502. At the distal end of the tubular member 502, the coolant fluid may pass into the second annular channel 508, via the connecting passageways in the separation wall 514. The coolant fluid may the flow along the second annular channel 508 back towards the proximal end of the tubular member 502. The coolant fluid may flow out of the second annular channel 508 via an outlet of the second annular channel 508 located near the proximal end of the tubular member 502. As the coolant fluid flows along the first annular channel 506 and second annular channel 508, heat may be removed from the tubular member 502 (and hence from an electrosurgical instrument received in the lumen 504). In an alternative configuration, the inlet connected to the second annular channel 508 and the outlet may be connected to the first annular channel 506, such that coolant fluid may be introduced from the coolant fluid source into the second annular channel 508, and the coolant fluid may exit via the first annular channel 506.

As the first and second annular channels 506, 508 are disposed concentrically around the lumen 504, heat may be removed from the tubular member 502 by the coolant fluid in a substantially uniform manner about a longitudinal axis of the tubular member 502.

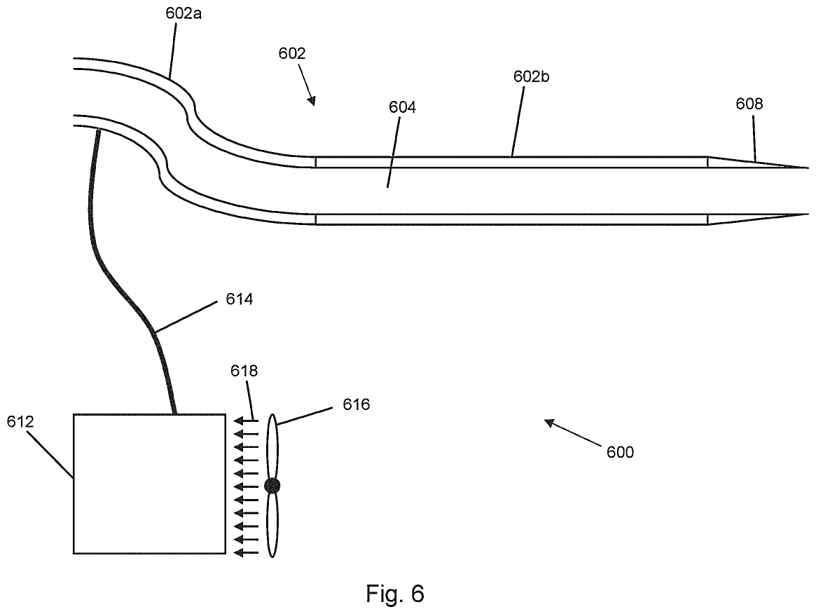

FIG. 6 shows a schematic diagram of an introducer 600 according to another embodiment of the invention. The introducer 600 includes a tubular member 602, which is formed of a proximal portion 602a and a distal portion 602b that are joined together. The tubular member 602 defines a lumen 604 through which an electrosurgical instrument is insertable. For illustration purposes, no electrosurgical instrument is shown in FIG. 6.

Both the proximal portion 602a and the distal portion 602b of the tubular member 602 are made of a thermally conductive material. The proximal portion 602a of the tubular member 602 is flexible (e.g. bendable and/or supple), whilst the distal portion 602b of the tubular member 602 is rigid. In particular, the distal portion 602b may be made of a material that has a greater stiffness than the proximal portion 602a. For example, the distal portion 602b may be formed by a hollow cylindrical metal tube (e.g. made of aluminium, copper, or brass), whilst the proximal portion 602a may be formed by a braided metal sleeve. The join between the proximal portion 602a and the distal portion 602b is configured to conduct heat, such that heat may flow between the proximal and distal portions. For example, the proximal portion 602a and the distal portion 602b may be welded together.

The lumen 604 extends through both the proximal and distal portions 602a, 602b of the tubular member, such that an electrosurgical instrument may be inserted through the tubular member 602. The lumen 604 is dimensioned such that when the electrosurgical instrument is inserted through the lumen 604, an outer surface of the electrosurgical instrument is in contact with a wall of the lumen 604. In this manner, the electrosurgical instrument may be thermally coupled to the tubular member 602 when it is inserted into the tubular member 602, such that heat may flow from the electrosurgical instrument to the tubular member 602.

A pointed distal end 608 is secured at a distal end of the distal portion 602b of the tubular member 602. The pointed distal end 608 may be made of a dielectric material, e.g. PEEK. The rigid distal portion 602b may facilitate percutaneous insertion of the distal portion 602b into a patient. The pointed distal end 608 may serve to pierce the patient's skin, to further facilitate percutaneous insertion. On the other hand, the flexible proximal portion 602a may enable a transmission line of the electrosurgical instrument to bend, which may facilitate handling of the electrosurgical instrument. For example, enabling the transmission line of the electrosurgical instrument to bend in the proximal portion 602a of the tubular member may facilitate connecting the transmission line to an electrosurgical generator.

The introducer 600 further includes a heat sink 612 which is thermally coupled to the proximal portion 602a of the tubular member 602 via a heat pipe 614. The introducer also includes a fan 616 which is configured to blow air onto the heat sink 612 (as shown by arrows 618), in order to actively cool the heat sink 612. The heat pipe 614, heat sink 612 and fan 616 may function in a similar manner to the heat pipe 214, heat sink 212 and fan 216 of introducer 200 described above. In this manner, heat from the tubular member 602 may flow into the heat sink 612 via the heat pipe 614, with the heat sink 612 being cooled by the fan 616. Heat from the distal portion 602b of the tubular member may flow into the proximal portion 602a, which is then removed via the heat pipe 614. As a result, the tubular member 602 may be maintained at a relatively low temperature so that damage to surrounding tissue may be avoided. This may also enable effective removal of heat from an electrosurgical instrument received in the lumen 604, such that the electrosurgical instrument may be maintained at a suitable working temperature.

In other examples, the heat sink 612 may be thermally coupled to the distal portion 602b instead of the proximal portion 602a. Together, the heat pipe 614, heat sink 612 and fan 616 may form a cooling assembly of the introducer 600.

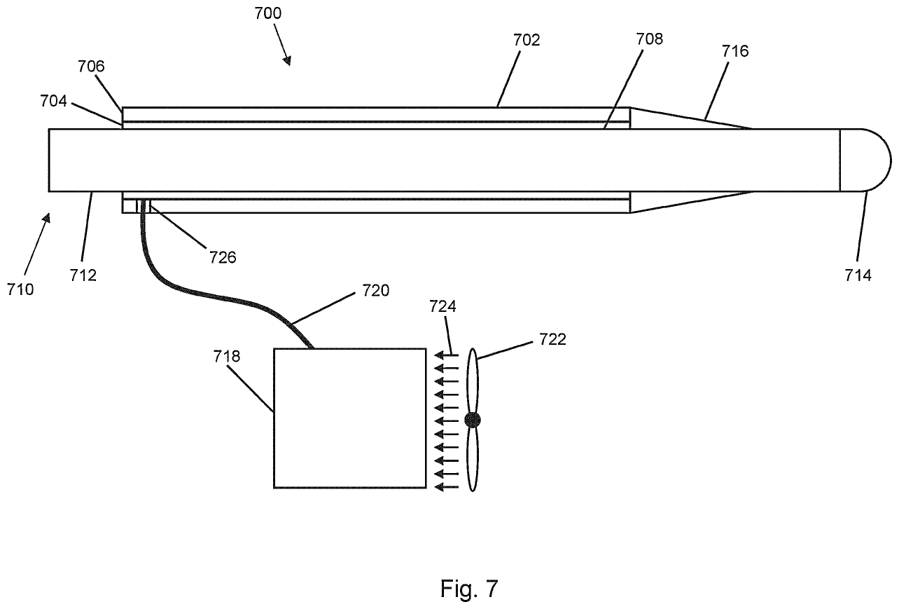

FIG. 7 shows a schematic diagram of an introducer 700 according to another embodiment of the invention. The introducer 700 is similar in configuration to introducer 200 described above, however a tubular member 702 of introducer 700 includes multiple layers made of different materials.

The tubular member 702 of introducer 700 includes an inner layer 704 that is concentric with an outer layer 706. The inner layer 704 is formed of a thermally conductive material, and the outer layer 706 is formed of a thermally insulating material. In one example, the outer layer 706 is formed by a hollow cylindrical tube of thermally insulating material, such as mica. The tube of thermally insulating material may have a wall thickness of approximately 0.2 mm. The inner layer 704 may then be formed as a coating of thermally conductive material (e.g. gold) that is deposited on an inner wall of the hollow cylindrical tube. The coating of thermally conductive material may have a thickness of approximately 0.05 mm, such that a total wall thickness of the tubular member 702 is 0.25 mm. A biocompatible coating may also be applied to an outer surface of the outer layer 706.

The tubular member 702 defines a lumen 708 through which an electrosurgical instrument 710 is insertable. The lumen 708 is defined by an inner surface of the inner layer 704. The lumen 708 is dimensioned such that when the electrosurgical instrument 710 is received within the lumen 708, an outer surface of the electrosurgical instrument 710 is in contact with the inner surface of the inner layer 704. For example, a cross-sectional area of the lumen 708 may match a cross-sectional area of the electrosurgical instrument 710. In this manner, the electrosurgical instrument 710 may be thermally coupled to the inner layer 704, so that heat may flow from the electrosurgical instrument 710 to the inner layer 704. The electrosurgical instrument 710 includes a transmission line 712 and radiating tip 714, and is similar in configuration to electrosurgical instrument 106 discussed above.

A pointed distal end 716 made of a dielectric material (e.g. PEEK) is disposed at a distal end of the tubular member 702. In some cases, the pointed distal end 716 may be made of the same material as the outer layer 706. For example, both the outer layer 706 and the pointed distal end 716 may be made of mica. In such an example, the pointed distal end 716 may be formed integrally with the outer layer 706.

The introducer 700 further includes a heat sink 718 which is thermally coupled to a proximal end of the tubular member 702 via a heat pipe 720. The introducer 700 also includes a fan 722 which is configured to blow air onto the heat sink 718 (as shown by arrows 724), in order to actively cool the heat sink 718. The heat pipe 720, heat sink 718 and fan 722 may function in a similar manner to the heat pipe 214, heat sink 212 and fan 216 of introducer 200 described above. In this manner, heat from the tubular member 702 may flow into the heat sink 718 via the heat pipe 720, with the heat sink 718 being cooled by the fan 722.

The heat pipe 720 is connected (i.e. thermally coupled) to the inner layer 704 of the tubular member 702, via a hole 726 formed in the outer layer 706. In this manner, heat may flow directly from the inner layer 704 to the heat sink 718 via the heat pipe 720, so that heat may be efficiently removed from the inner layer 704. The heat pipe 720 may also be connected to the outer layer 706, so that heat from both the inner layer 704 and the outer layer 706 may flow to the heat sink 718 via the heat pipe 720.

As a thermal conductivity of the inner layer 704 is greater than a thermal conductivity of the outer layer 706, heat may preferentially flow along the inner layer 704. In this manner, the outer layer 706 may act as a thermal barrier between the electrosurgical instrument 710 and surrounding tissue. Thus, tubular member 702 may enable heat from the electrosurgical instrument 710 to be effectively removed via the inner layer 704 of the tubular member 702, whilst minimising heating of surrounding tissue. The concept of a tubular member having a thermally conductive inner layer and a thermally insulating outer layer may be applied to any of the other embodiments described herein.

Together, the electrosurgical instrument 710 and introducer 700 may form part of an electrosurgical system that is an embodiment of the invention.

In the embodiments discussed above, various configurations of cooling assembly have been described. In further embodiments, features of the various cooling assemblies discussed above may be combined, in order to further improve heat removal from the tubular member.

In the embodiments discussed above, the introducers may be used for percutaneous procedures, e.g. where the tubular member of the introducer is inserted percutaneously into the body of a patient. However, the above embodiments may be adapted so that they are suitable for use with a surgical scoping device, such as a laparoscope. For example, the tubular member of the introducer in the above embodiments may be dimensioned such that it fits in a working channel of the surgical scoping device. Additionally, the tubular member may be provided without a pointed distal end, in order to avoid damage to the surgical scoping device.

1. An introducer for introducing an electrosurgical instrument into a body of a patient, the introducer comprising:
a tubular member defining a lumen through which the electrosurgical instrument is insertable; and
a cooling assembly configured to remove heat from the tubular member;
wherein the cooling assembly includes a heat sink that is thermally coupled to the tubular member, the heat sink being of a the having a greater heat capacity than the tubular member.

2. An introducer according to claim 1, wherein the heat sink is disposed at or near a proximal end of the tubular member.

3. An introducer according to claim 1, wherein the heat sink is disposed in a handle of the tubular member.

4. An introducer according to claim 1, wherein the heat sink is thermally coupled to the tubular member via a heat pipe.

5. An introducer according to claim 1, wherein the cooling assembly is configured to actively cool the heat sink.

6. An introducer according to claim 5, wherein the cooling assembly includes a fan configured to actively cool the heat sink.

7. An introducer according to claim 5, wherein the cooling assembly is configured to actively cool the heat sink with a coolant fluid.

8. An introducer according to claim 5, wherein the cooling assembly includes a heat exchanger configured to actively cool the heat sink.

9. An introducer according to claim 1, wherein the cooling assembly includes a heat pump configured to remove heat from the tubular member.

10. An introducer according to claim 1,
wherein the tubular member includes one or more channels defined on or in a sidewall of the tubular member, and wherein the cooling assembly is configured to circulate a coolant fluid through the one or more channels to remove heat from the tubular member.

11. An introducer according to claim 1, wherein the tubular member includes one or more contact elements disposed within the lumen and arranged to press against an outer surface of the electrosurgical instrument when the electrosurgical instrument is inserted through the lumen.

12. An introducer according to claim 1, wherein the tubular member includes a distal end formed of a dielectric material.

13. An introducer according to claim 1, wherein the tubular member includes a pointed distal end.

14. An introducer according to claim 1, wherein the tubular member includes an outer layer formed of a biocompatible material.

15. An introducer according to claim 1, wherein the tubular member includes an inner layer formed of a thermally conductive material, and an outer layer formed of a thermally insulating material.

16. An introducer according to claim 1, wherein a proximal portion of the tubular member is flexible, and a distal portion of the tubular member is rigid.

17. An introducer according to claim 1, wherein a length of the tubular member is 30 cm or greater.

18. An electrosurgical system comprising:
an electrosurgical instrument comprising:
a transmission line for conveying microwave and/or radiofrequency electromagnetic (EM) energy; and
a radiating tip mounted at a distal end of the transmission line configured to receive and deliver the microwave and/or radiofrequency EM energy to biological tissue; and
an introducer according to claim 1,
wherein the electrosurgical instrument is insertable through the lumen of the tubular member.

19. A method of introducing an electrosurgical instrument into a body of a patient, the method comprising:
inserting a tubular member of an introducer into the body of the patient;
inserting an electrosurgical instrument through a lumen of the tubular member, such that a radiating tip of the electrosurgical instrument protrudes beyond a distal end of the tubular member; and
removing heat from the tubular member using a cooling assembly of the introducer, wherein the cooling assembly includes a heat sink that is thermally coupled to the tubular member, the heat sink being formed of a thermally conductive material and having a greater heat capacity than the tubular member.
